(19) United States
(12) Patent Application Publication
(10) Pub. No.: US 2016/0327573 A1
(43) Pub. Date: Nov. 10, 2016

(54) Methods for Diagnosing Onset and Monitoring Progression of Huntington's Disease

(71) Applicant: Emory University, Atlanta, GA (US)

(72) Inventors: Anthony Wing Sang Chan, Atlanta, GA (US); Melinda Sue Prucha, Decatur, GA (US)

(21) Appl. No.: 15/148,498

(22) Filed: May 6, 2016

Related U.S. Application Data
(60) Provisional application No. 62/158,584, filed on May 8, 2015.

(51) Int. Cl. G01N 33/68; G01N 33/50; G01N 33/84; G01N 33/64; G01N 33/82; G01N 33/74

(52) U.S. Cl. CPC G01N 33/6896; G01N 33/6812; G01N 33/6815; G01N 33/82; G01N 33/743; G01N 33/84; G01N 33/64; G01N 33/50; G01N 2570/00; G01N 2560/00; G01N 2800/2835; G01N 2800/56

(57) ABSTRACT

The present invention relates to methods of diagnosing onset, monitoring progression and initiating therapies for a neurological disorder, such as juvenile- or adult-onset Huntington's disease, by comparing the levels of one or more plasma and/or cerebrospinal fluid biomarkers. The invention further relates to N-methyl proline, beta-tocopherol, cortisone, pyruvate, phosphate, and trans-4-hydroxyproline as plasma and/or cerebrospinal fluid biomarkers of onset and progression of a neurological disorder, such as Huntington's disease.

### CROSS REFERENCE TO RELATED APPLICATIONS

This application claims the benefit of priority to U.S. Provisional Application No. 62/158,584 filed May 8, 2015. The entirety of this application is hereby incorporated by reference for all purposes.

### STATEMENT REGARDING FEDERALLY SPONSORED RESEARCH OR DEVELOPMENT

This invention was made with government support under Grant R24OD010930 awarded by the National Institutes of Health. The government has certain rights in the invention.

### BACKGROUND

Huntington's disease (HD) is a fatal autosomal-dominant neurodegenerative disorder characterized by onset and progressive decline in motor function and cognitive abilities. In majority of cases, the age of onset is around 40 years, but is highly variable. About 5-10% of HD patients will have an onset before 20 years of age (juvenile HD), while about 20% of HD individuals will develop the disease after 50 years of age (Myers R H, et al. Late onset of Huntington's disease. J Neurol Neurosurg Psychiatry. 1985; 48:530-534).

HD is caused by an expansion of CAG trinucleotide repeat region of the gene encoding the huntingtin protein (HTT). In healthy subjects, the number of CAG repeats in HTT gene is up to 35. Individuals with HD have an increased number of the CAG repeat from 36 to 120, and the higher the number of the repeats, the earlier is the age of onset of disease. While the genetic tests can identify individuals with expanded number of the trinucleotide repeat, the age of onset of HD for these individuals, and the rate of progression of disease, are highly variable. The age of onset can vary by 20 years in the most common number of repeat expansion, which is 45 to 50 CAG repeats, and can begin at 35 years of age. The age of onset of HD in individuals with lower than 45 repeats can vary as broadly as from 30 to 90 years of age (Brinkman R R, et al. The likelihood of being affected with Huntington disease by a particular age, for a specific CAG size. Am J Hum Genet. 1997; May; 60(5):1202-10; WO 02/29408 A2).

Current therapeutic options only target the alleviation of symptoms; there is no cure. More recently, the research community has focused on the pre-manifest stages of HD (prodromal-HD), prior to onset of clinical signs of HD when therapeutic intervention might have the highest efficacy for preserving motor function and cognitive abilities. Findings from TRACK-HD and PREDICT-HD, longitudinal observational studies aimed at identifying clinical manifestations of early HD, have shown measureable differences in motor and cognitive function prior to HD onset and during disease progression (Paulsen J S, et al. Detection of Huntington's disease decades before diagnosis: the Predict-HD study. J. Neurol. Neurosurg. Psychiatry. 2008; 79, 874-880; Tabrizi S J, et al. Biological and clinical manifestations of Huntington's disease in the longitudinal TRACK-HD study: cross-sectional analysis of baseline data. Lancet Neurol. 2009; 8, 791-801; Tabrizi S J, et al. Biological and clinical changes in premanifest and early stage Huntington's disease in the TRACK-HD study: the 12-month longitudinal analysis. Lancet Neurol. 2011; 10, 31-42; Tabrizi S J, et al. Potential endpoints for clinical trials in premanifest and early Huntington's disease in the TRACK-HD study: analysis of 24 month observational data. Lancet Neurol. 2012; 11, 42-53). These studies indicated that clinical symptoms of the neurodegenerative process occur well in advance of the diagnosis of onset of HD. However, these clinical measurements can be insensitive to changes occurring in prodromal HD (Andre R, et al. Biomarker development for Huntington's disease. Drug Discov Today. 2014; pii: S1359-6446(14)00079-8). Therefore, metabolic biomarkers, which are easier and cheaper to obtain and analyze, and which will change longitudinally with disease progression will be extremely valuable to the field.

Metabolomics profiling studies are being used more and more in pursuit of novel disease biomarkers for HD (Casseb R F, et al. Thalamic metabolic abnormalities in patients with Huntington's disease measured by magnetic resonance spectroscopy. Braz J Med Biol Res. 2013; 46(8):722-727. Eckhart A D, et al. Metabolomics as a key integrator for “omic” advancement of personalized medicine and future therapies. Clin Transl Sci. 2012 June; 5(3):285-8. Lee D Y, et al. Mass spectrometry-based metabolomics, analysis of metabolite-protein interactions, and imaging. Biotechniques. 2010 August; 49(2):557-65; Quinones and Kaddurah-Daouk. Metabolomics tools for identifying biomarkers for neuropsychiatric diseases. Neurobiol Dis. 2009 August; 35(2):165-76). Several reported metabolomics profiling studies have used different HD animal models; however, these studies were based largely on cross-sectional, not longitudinal, analysis of individuals. Cross-sectional studies are limited in that it is often difficult to control for environmental variables and truly isolate individual risk factors that inevitably play a role in HD onset and progression.

As current outcome measures are relatively insensitive in prodromal-HD patients for predicting the onset of HD (Andre R, et al. Biomarker development for Huntington's disease. Drug Discov Today. 2014; pii: S1359-6446(14)00079-8), there remains a particular need for identifying biomarkers of onset of HD prior to manifestations of the clinical signs for the timely delivery of therapeutic interventions and preservation of motor function and cognitive abilities.

### SUMMARY

The present invention relates to the methods of diagnosing onset and monitoring progression of neurological disorders, such as juvenile or adult Huntington's disease (HD) by measuring and monitoring levels of plasma and CSF biomarkers. The invention also relates to methods of initiation of therapies for HD in a subject with biomarker levels indicative of onset of HD. The invention further relates to methods of treating a subject with biomarker levels indicative of progression of HD with a HD therapy for progression of HD.

In some embodiments, the methods relate to diagnosing the onset of HD and comprise obtaining a sample from a subject, measuring the levels of at least one or more, two or more, three more, four or more, five or more, six or more, seven or more, eight or more, or nine or more biomarkers in said sample, comparing the levels of said biomarkers to reference levels or normal levels, e.g., those observed in healthy subjects, diagnosing said subject with an onset of HD when the levels of said biomarkers from said subject differ from the reference or normal levels, e.g., those observed in healthy subjects, and treating said subject with a HD therapy. In one embodiment, said sample is plasma and/or CSF, said subject is human, and said biomarkers comprise N-methyl proline and beta-tocopherol, or N-methyl proline, beta-tocopherol and cortisone, or beta-tocopherol, pyruvate, phosphate and trans-4-hydroxyproline. In other embodiments, said sample is plasma and/or CSF, said subject is human, said plasma biomarkers comprise N-methyl proline, cortisone, beta-tocopherol, 2-hydroxyisobutyrate, trans-4-hyroxyproline, butyrylglicine, phosphate, pyruvate, or any number of combinations thereof, and said CSF biomarkers comprise cortisone, asparagine, histidine, homostachydrine, methionine, pantothenate, phenylalanine, tyrosine, and uridine, or any number of combinations thereof. In another embodiment, said biomarkers of onset of HD are any one or any combination of biomarkers reported herein. The different levels of said plasma and CSF biomarkers used to diagnose the onset of HD are disclosed herein

In other embodiments, the methods relate to monitoring progression of HD and comprise obtaining at least two serial samples from a subject, measuring the levels of at least one or more, two or more, three or more, or four or more biomarkers in said samples, comparing the levels of said biomarkers to a normal or reference value, e.g., to those observed in healthy subjects, comparing the levels of said biomarkers from the serial samples obtained from said subject, and detecting progression of HD in said subject when the levels of said biomarkers in the latest sample from said subject differ from levels in preceding samples obtained from the same subject, and treating said subject with a HD therapy for progression of HD. In one embodiment, said sample is plasma, said subject is a human, said serial samples are samples collected from the same subject at two or more time points separated by at least one or more, two or more, three or more, four or more, five or more six or more months. In one embodiment, said plasma biomarkers comprise beta-tocopherol, pyruvate, phosphate and trans-4-hydroxyproline. In other embodiments, said plasma biomarkers are at least one or more, two or more, three or more, four or more, five or more, six or more, seven or more, or eight or more biomarkers selected from N-methyl proline, beta-tocopherol, cortisone, 2-hydroxyisobutyrate, trans-4-hyroxyproline, butyrylglicine, phosphate, pyruvate, or any number of combinations thereof. In another embodiment, said biomarkers of progression of HD are any one or any combination of biomarkers selected from those reported herein. The different levels of said plasma biomarkers are those reported herein.

No treatments available today can cure HD, but medications can lessen some of the movement and psychiatric symptoms associated with the disease. A combination of multiple therapies can help a person adapt to changes in his or her abilities for a certain amount of time.

There are advantages associated with early detection of onset of HD, or non-invasive monitoring of progression of HD, to help select treatment options for HD patients. During selection of therapies, medication management is likely to evolve over the course of the disease, depending on the overall treatment goals and course of disease progression. Metabolomic information, indicative of onset and/or progression of the disease, may help select treatment options that offer maximal benefit with the lowest side effect and improve quality of care.

### BRIEF DESCRIPTION OF THE FIGURES

FIG. 1A shows data for a barrier/detour task. Barrier/detour task was performed at 8 months of age. Scores are latency (sec) to retrieve the reward. Data were collected from HD (Huntington's disease) and WT (wild-type control) monkeys (n=4 per group). rHDs: rHD6, rHD7 and rHD8. White is WT, grey rHD1, and black is rHDs.

FIG. 1B shows data for a barrier/detour task. Barrier/detour task was performed at 8 months of age. Scores are frequency of reaching through the transparent plastic barrier. Data were collected from HD (Huntington's disease) and WT (wild-type control) monkeys (n=4 per group). rHDs: rHD6, rHD7 and rHD8.

FIG. 1C shows data for a barrier/detour task. Barrier/detour task was performed at 8 months of age. Scores are number of motor problems. Data were collected from HD (Huntington's disease) and WT (wild-type control) monkeys (n=4 per group). rHDs: rHD6, rHD7 and rHD8.

FIG. 1D shows data for a barrier/detour task. Barrier/detour task was performed at 8 months of age. Scores are frequency of reaching repetitively through the transparent wall opposite to the opening. Data were collected from HD (Huntington's disease) and WT (wild-type control) monkeys (n=4 per group). rHDs: rHD6, rHD7 and rHD8.

FIG. 1E shows data for a barrier/detour task. Barrier/detour task was performed at 16 months of age. Scores are latency (sec) to retrieve the reward. Data were collected from HD (Huntington's disease) and WT (wild-type control) monkeys (n=4 per group). # indicates near significance (p=0.07). rHDs: rHD6, rHD7 and rHD8.

FIG. 1F shows data for a barrier/detour task. Barrier/detour task was performed at 16 months of age. Scores are frequency of reaching through the transparent plastic barrier. Data were collected from HD (Huntington's disease) and WT (wild-type control) monkeys (n=4 per group). * indicates a p-value <0.05. rHDs: rHD6, rHD7 and rHD8.

FIG. 1G shows data for a barrier/detour task. Barrier/detour task was performed at 16 months of age. Scores are number of motor problems. Data were collected from HD (Huntington's disease) and WT (wild-type control) monkeys (n=4 per group). * indicates a p-value <0.05. rHDs: rHD6, rHD7 and rHD8.

FIG. 1H shows data for a barrier/detour task. Barrier/detour task was performed at 16 months of age. Scores are frequency of reaching repetitively through the transparent wall opposite to the opening. Data were collected from HD (Huntington's disease) and WT (wild-type control) monkeys (n=4 per group). # indicates near significance (p=0.07). rHDs: rHD6, rHD7 and rHD8.

FIG. 2 shows a visuospatial orientation (Life Saver) task at 16 and 36 months of age. Life Saver task was performed at 16 (left panel) and 36 (right panel) months of age. Latency (sec) and fastest times (sec) to free a ring-shaped candy (“Life Saver”) by moving it along metal rods of increasing difficulty. Easy rods contained 1-3 bends and difficult rods contained 4-5 bends. Please note that testing of rHD1 in the Life Saver task had to be discontinued due to the development of self-injury behavior during testing. * indicates a significant difference between HD (Huntington's disease) and WT (wild-type control) monkeys (p<0.05). rHDs: rHD6, rHD7 and rHD8.

FIG. 3 shows a timeline of progressive development of behavioral impairment of HD monkeys. Over the first several years of age, behavior was monitored in the HD monkeys; specific types of impairment are plotted on a timeline for rHDs 6, 7 and 8 (top) and rHD1 (bottom).

FIG. 4 shows data CSF metabolite levels in HD vs. WT monkeys. The complete CSF dataset and the enriched subset of 9 significant metabolites were subjected to principal components analysis (PCA). The first 3 principal components (Prins) are plotted in 3-D scatterplots. Ellipsoids highlight disease status (HD; WT). Individual metabolite levels were measured in a cross-sectional analysis at 24 months of age. Data are plotted as mean HD (box) versus mean WT (box). No gender-based effect (WT only) or construct-specific effect (HD only) was detectable; however, means for each gender and construct are plotted for visualization. * denotes a significant difference between HD and WT mean metabolite levels at 24 months (p<0.05).

FIG. 5 shows data for metabolite biomarker candidates for HD. There were 8 metabolites found to be highly significant based on results from the linear mixed-effect statistical model (p<0.01, HD, HD□time) spanning data generated at 6-month intervals longitudinally over 2 years in HD and WT monkeys. Data for the 8 candidates were partitioned by age prior to PCA analysis. Ellipsoids encompassing disease status (HD; WT) were superimposed in 3-D scatterplots of results for each time point. Each biomarker candidate was plotted independently, displaying biomarker potential. Data plotted are representative of mean HD (triangles) versus mean WT (circles) over time (5, 11, 17 and 23 months age). Trends are depicted by dashed lines displaying a fit binomial regression curve for each candidate. To highlight any potential gender effect (WT only) or construct effect (HD only), means for each gender and each construct were plotted. Joint p-values (HD, HD□time) are displayed for each candidate. * denotes a stronger, more significant trend was correlated with the group of rHD monkeys with the pHtt construct (though overall mean p-value is reported for all). # denotes a significant gender effect between WT males and females at denoted age (p<0.05).

FIG. 6A shows data for Infant Neurobehavioral Assessment Scale (INAS)—Rating scores for orientation responses spanning the first 5 weeks of postnatal age HD animals (rHD1, rHD6, rHD7, rHD8) and WT control animals (WT; n=3). * indicate significant differences between HD and WT monkeys.

FIG. 6B shows data for Infant Neurobehavioral Assessment Scale (INAS)—Rating scores for neuromotor responses spanning the first 5 weeks of postnatal age HD animals (rHD1, rHD6, rHD7, rHD8) and WT control animals (WT; n=3). * indicate significant differences between HD and WT monkeys.

FIG. 6C shows data for Infant Neurobehavioral Assessment Scale (INAS)—Rating scores for temperament measures spanning the first 5 weeks of postnatal age HD animals (rHD1, rHD6, rHD7, rHD8) and WT control animals (WT; n=3).

FIG. 6D shows data for Infant Neurobehavioral Assessment Scale (INAS)—Rating scores for motor activities spanning the first 5 weeks of postnatal age HD animals (rHD1, rHD6, rHD7, rHD8) and WT control animals (WT; n=3). * indicate significant differences between HD and WT monkeys.

FIG. 7A shows data for Juvenile Neurobehavioral Assessment Scale (JNAS)—Rating scores for motor activity observed weekly from 1 to 6 months of age in HD animals (rHD1, rHD6, rHD7, rHD8) and 3 WT control monkeys (WT).

FIG. 7B shows data for Juvenile Neurobehavioral Assessment Scale (JNAS)—Rating scores for neuromotor responses observed weekly from 1 to 6 months of age in HD animals (rHD1, rHD6, rHD7, rHD8) and 3 WT control monkeys (WT).

FIG. 8 schematically illustrates methods for metabolomics profiling. Plasma samples were collected longitudinally at 6-month intervals from HD (n=4-5) monkeys and WT (n=4) age-matched control monkeys through the first 2 years of age. CSF was collected at 2 years of age. All samples were processed for large-scale metabolomics profiling by mass spectrometry. Statistical analysis was performed to identify HD biomarkers in the plasma and CSF samples.

### DETAILED DISCUSSION

The following detailed description is intended to illustrate the various embodiments of methods for diagnosing onset and monitoring progression of Huntington's disease using biomarkers described in the disclosure. As such, this detailed description is not meant to be limiting of the scope or application of the embodiments listed herein. It will be understood by persons skilled in the art that numerous modifications, substitutions, changes, or replacements with equivalents may be made to the particulars of the disclosure without altering the scope of the embodiments, and that such equivalents are to be included herein.

### DEFINITIONS

As used herein, the term “diagnosing” refers to determining whether the relevant disease is present or absent. It also refers to, in relation to HD, determining whether the disease is juvenile HD, or adult HD. The term “diagnosing” also relates to determining whether the disease is in prodromal or manifest stages of juvenile or adult HD.

As used herein, the term “onset” refers to an initiation in decline of motor and cognitive functions associated with neurodegeneration. It also refers to, in relation to HD, an initiation in changes in any one or more, or a combination of any two or more, molecular biomarkers for HD selected from those reported herein.

As used herein, the term “prodromal” relates to a stage in HD pathogenesis, at which subjects who were identified to have expanded CAG repeats in the HTT gene do not have sufficient motor or cognitive deficits to be diagnosed with having HD, and who may have non-specific early cognitive or psychiatric symptoms (“prodromal-HD”).

As used herein, the term “sample” refers to a variety of sample types obtained from an individual that can be used in a diagnostic or monitoring assay and includes, but is not limited to, blood (including whole blood), plasma or serum, urine, cerebrospinal fluid, tears or saliva. The term also includes samples that have been manipulated in any way after their procurement, such as by treatment with reagents, solubilization, or enrichment for certain components, such as proteins or polynucleotides.

As used herein, the term “subject” refers to any animal, preferably a human patient, livestock, or domestic pet.

As used herein, the term “subject with onset of HD” refers to a subject who is in a process of developing clinical symptoms and/or biomolecular signature of HD. A genetic test determining the number of CAG repeats in the HTT gene may also be performed to identify the subject as being at risk of developing HD. The term “clinical symptoms of HD” as used herein refers to motor and cognitive deficits and are detected through assessment by a physician. The term “biomolecular signature” as used herein refers to changes in any one or more, or a combination of any two or more, molecular biomarkers for HD selected from those reported herein.

As used herein, the term “healthy subject” refers to an individual who has not, or would be assessed by a physician as not having, HD or other neurological condition. A healthy subject is typically age-matched within a range of 5 to 10 years, including, but not limited to, an individual that is age-matched with the individual to be assessed.

As used herein, the term “biomarker” refers to compounds used for aiding diagnosis of onset of symptoms and monitoring of progression of neurological disorder, such as HD. The term also refers to compounds used for predicting a future (within the next one to twenty (20) or more years) decline in the state of motor and cognitive functions associated with neurodegeneration. The term “biomarker” refers to a marker of disease state that determines the severity of disease and may change as the disease progresses or in response to treatment.

As used herein, the term “neurological disorder” typically refer to a disease or disorder of the central nervous system. Neurological disorders or diseases include, but are not limited to multiple sclerosis, neuropathies, and neurodegenerative disorders such as Alzheimer's disease (AD), Huntington's disease, Parkinson's disease, amyotrophic lateral sclerosis (ALS), mild cognitive impairment (MCI), Downs's and all forms of dementia including front temporal dementia, Dementia with Lewy Bodies, Vascular dementia, and Parkinson's disease dementia.

As used herein, the terms “therapy”, “treatment” or “treating” are not limited to the case where the subject (e.g. patient) is cured and the disease is eradicated. Rather, embodiments of the present disclosure also contemplate treatment that merely reduces symptoms, and/or delays disease progression. For example, the treatment may alleviate, ameliorate, and/or stabilize symptoms, as well as delay progression of symptoms, of a particular disorder.

As used herein, the term “levels” refers to amount, concentration, relative amount, and/or relative concentration of a substance in a sample from a subject. The amount and/or concentration are measured through quantitative assays, including, but not limited to, mass spectrometry, enzyme-linked immunosorbent assay (ELISA) and high-performance liquid chromatography (HPLC).

As used herein, the term “progression” refers to advancement in the decline of motor and cognitive functions of the subject diagnosed with a neurologic disorder. The term also refers to changes in any one or more, or a combination of any two or more, molecular biomarkers for HD selected from those reported herein.

As used herein, the term “serial samples” refers to consecutive samples obtained one after another from the same subject, but separated with a period of time, such as one or more days, months, or years.

As used herein, the term “months” refers to calendar months, or time intervals of about 28 to 31 days.

### Methods for Diagnosing Onset and Monitoring Progression of HD

The present invention relates to the methods of diagnosing onset and monitoring progression of neurological disorders, such as juvenile or adult HD, by measuring and monitoring levels of plasma and CSF biomarkers. The invention also relates to methods of initiation of therapies for HD in a subject with biomarker levels indicative of onset of HD. The invention further relates to methods of treating a subject with biomarker levels indicative of progression of HD with a HD therapy for progression of HD.

In one embodiment, the methods relate to diagnosing the onset of HD and comprise obtaining a sample from a subject, measuring the levels of at least one or more, or two or more biomarkers in said sample, comparing the levels of said biomarkers to reference or normal level, e.g., to those observed in healthy subjects, diagnosing said subject with an onset of HD when the levels of said biomarkers from said subject differ from those observed in healthy subjects, and treating said subject with a HD therapy. In one embodiment, said sample is plasma, said subject is human, said biomarkers comprise N-methyl proline and beta-tocopherol. Specifically, said difference in the levels of said biomarkers is a lower level of N-methyl proline in subjects with onset of HD when compared to that of healthy subjects. Said lower level of plasma N-methyl proline is about 5.8 raw peak intensity (log 10) for subjects with suspected onset of HD and is about 6.20 raw peak intensity (log 10) for healthy subjects. Said level of beta-tocopherol is ≧4.30 raw peak intensity (log 10) for subjects with suspected onset of HD and is ≦4.7 raw peak intensity (log 10) for healthy subjects.

In other embodiments, the methods relate to diagnosing the onset of HD and comprise obtaining a sample from a subject, measuring the levels of at least one or more, two or more, or three or more biomarkers in said sample, comparing the levels of said biomarkers to reference or normal levels, e.g., to those observed in healthy subjects, diagnosing said subject with an onset of HD when the levels of said biomarkers from said subject differ from those observed in healthy subjects, and treating said subject with a HD therapy. In one embodiment, said sample is plasma and/or CSF, said subject is human, said biomarkers comprise N-methyl proline, beta-tocopherol and cortisone. Specifically, said difference in the levels of said biomarkers is a lower level of N-methyl proline and cortisone biomarkers in subjects with onset of HD when compared to that of healthy subjects. Said lower level of plasma N-methyl proline is about 5.8 raw peak intensity (log 10) for subjects with suspected onset of HD and is about 6.20 raw peak intensity (log 10) for healthy subjects. Said level of beta-tocopherol is ≧4.30 raw peak intensity (log 10) for subjects with suspected onset of HD and is ≦4.7 raw peak intensity (log 10) for healthy subjects. Said lower level of plasma cortisone is from about 5.35 to 5.45 raw peak intensity (log 10) for subjects with suspected onset of HD and is about 5.55 raw peak intensity (log 10) for healthy subjects. Said lower level of CSF cortisone is about 4.9 raw peak intensity (log 10) for subjects with suspected onset of HD and is about 5 raw peak intensity (log 10) for healthy subjects.

In other embodiments, the methods relate to diagnosing the onset of HD and comprise obtaining a sample from a subject, measuring the levels of at least one or more, two or more, three or more, or four or more biomarkers in said sample, comparing the levels of said biomarkers to reference or normal levels, e.g., to those observed in healthy subjects, diagnosing said subject with an onset of HD when the levels of said biomarkers from said subject differ from those observed in healthy subjects, and treating said subject with a HD therapy. In one embodiment, said sample is plasma, said subject is human, said biomarkers comprise beta-tocopherol, pyruvate, phosphate and trans-4-hydroxyproline. Specifically, said differences in the levels of said biomarkers are: beta-tocopherol is ≧4.30 raw peak intensity (log 10) for subjects with suspected onset of HD and is ≦4.7 raw peak intensity (log 10) for healthy subjects, pyruvate is about 5.7 to 5.9 raw peak intensity (log 10) for subjects with suspected onset of HD and about 5.7 to 6.4 raw peak intensity (log 10) for healthy subjects, phosphate is about 7.93 to 8.6 raw peak intensity (log 10) for subjects with suspected onset of HD and about 8 to 8.18 raw peak intensity (log 10) for healthy subjects, and trans-4-hydroxyproline is about 6.8 raw peak intensity (log 10) for subjects with suspected onset of HD and about 6.55 to 6.95 raw peak intensity (log 10) for healthy subjects.

In other embodiments, the methods relate to diagnosing the onset of HD and comprise obtaining a sample from a subject, measuring the levels of at least one or more, two or more, three or more, four or more, five or more, six or more, seven or more, eight or more, and nine or more biomarkers in said sample, comparing the levels of said biomarkers to reference or normal levels, e.g., to those observed in healthy subjects, diagnosing said subject with an onset of HD when the levels of said biomarkers from said subject differ from those observed in healthy subjects, and treating said subject with a HD therapy. In one embodiment, said sample is plasma and/or CSF, said subject is human, said plasma biomarkers comprise N-methyl proline, cortisone, beta-tocopherol, trans-4-hyroxyproline, 2-hydroxyisobutyrate, butyrylglicine, phosphate, pyruvate, or any number of combinations thereof, and said CSF biomarkers comprise cortisone, asparagine, histidine, homostachydrine, methionine, pantothenate, phenylalanine, tyrosine, and uridine, or any number of combinations thereof. Specifically, said differences in the levels of said plasma biomarkers are: N-methyl proline is about 5.8 raw peak intensity (log 10) for subjects with suspected onset of HD and is about 6.20 raw peak intensity (log 10) for healthy subjects, beta-tocopherol is ≧4.30 raw peak intensity (log 10) for subjects with suspected onset of HD and is ≦4.7 raw peak intensity (log 10) for healthy subjects, plasma cortisone is from about 5.35 to 5.45 raw peak intensity (log 10) for subjects with suspected onset of HD and is about 5.55 raw peak intensity (log 10) for healthy subjects, trans-4-hyroxyproline is about 6.8 raw peak intensity (log 10) for subjects with suspected onset of HD and about 6.55 to 6.95 raw peak intensity (log 10) for healthy subjects, 2-hydroxyisobutyrate is about 5.7 raw peak intensity (log 10) for subjects with suspected onset of HD and is about ≧5.65 raw peak intensity (log 10) for healthy subjects, butyrylglicine is about ≦5.2 raw peak intensity (log 10) for subjects with suspected onset of HD and is about 4.8 65 raw peak intensity (log 10) for healthy subjects, phosphate is about phosphate is about 7.93 to 8.6 raw peak intensity (log 10) for subjects with suspected onset of HD and about 8 to 8.18 raw peak intensity (log 10) for healthy subject, and pyruvate is about 5.7 to 5.9 raw peak intensity (log 10) for subjects with suspected onset of HD and about 5.7 to 6.4 raw peak intensity (log 10) for healthy subjects. Specifically, said differences in the levels of said CSF biomarkers are: CSF cortisone is about 4.9 raw peak intensity (log 10) for subjects with suspected onset of HD and is about 5 raw peak intensity (log 10) for healthy subjects, asparagine is about 5.65 to 5.75 raw peak intensity (log 10) for subjects with suspected onset of HD and is about 5.55 to 5.50 raw peak intensity (log 10) for healthy subjects, histidine is about 5.10 raw peak intensity (log 10) for subjects with suspected onset of HD and is about 4.9 raw peak intensity (log 10) for healthy subjects, homostachydrine is about 4.8 raw peak intensity (log 10) for subjects with suspected onset of HD and is about 5.8 raw peak intensity (log 10) for healthy subjects, methionine is about 5.45 to 5.6 raw peak intensity (log 10) for subjects with suspected onset of HD and is about 5.3 raw peak intensity (log 10) for healthy subjects, pantothenate is about 5.4 raw peak intensity (log 10) for subjects with suspected onset of HD and is about 5.3 to 5.35 raw peak intensity (log 10) for healthy subjects, phenylalanine is about 6.7 to 6.8 raw peak intensity (log 10) for subjects with suspected onset of HD and is about 6.65 raw peak intensity (log 10) for healthy subjects, tyrosine is about 6.5 to 6.6 raw peak intensity (log 10) for subjects with suspected onset of HD and is about 6.3 to 6.4 raw peak intensity (log 10) for healthy subjects, and uridine is about 5.25 to 5.358 raw peak intensity (log 10) for subjects with suspected onset of HD and is about 5.1 to 5.2 raw peak intensity (log 10) for healthy subjects.

In other embodiments, said plasma and CSF biomarkers of onset of HD are any one or any combination of biomarkers selected from those reported herein. The different levels of said plasma and CSF biomarkers used to diagnose the onset of HD are those reported herein.

In another embodiment, the methods relate to monitoring progression of HD and comprise obtaining at least two serial samples from a subject, measuring the levels of at least one or more, two or more, three or more, or four or more biomarkers in said samples, comparing the levels of said biomarkers to reference or normal levels, e.g., to those observed in healthy subjects, comparing the levels of said biomarkers from the serial samples obtained from said subject, and detecting progression of HD in said subject when the levels of said biomarkers in the latest sample from said subject differ from levels in preceding samples obtained from the same subject, and treating said subject with a HD therapy for progression of HD. In one embodiment, said sample is plasma, said subject is a human, said serial samples are samples collected from the same subject at two or more time points separated by at least one or more, two or more, three or more, four or more, five or more six or more months. In one embodiment, said plasma biomarkers comprise beta-tocopherol, pyruvate, phosphate and trans-4-hydroxyproline. Specifically, said difference in the levels of said biomarkers are: beta-tocopherol is ≧4.30 raw peak intensity (log 10) for subjects with suspected onset of HD and is ≦4.7 raw peak intensity (log 10) for healthy subjects, pyruvate is about 5.7 to 5.9 raw peak intensity (log 10) for subjects with suspected onset of HD and about 5.7 to 6.4 raw peak intensity (log 10) for healthy subjects, phosphate is about 7.93 to 8.6 raw peak intensity (log 10) for subjects with suspected onset of HD and about 8 to 8.18 raw peak intensity (log 10) for healthy subjects, and trans-4-hydroxyproline is about 6.8 raw peak intensity (log 10) for subjects with suspected onset of HD and about 6.55 to 6.95 raw peak intensity (log 10) for healthy subjects.

In another embodiment, the methods relate to monitoring progression of HD and comprise obtaining at least two serial samples from a subject, measuring the levels of at least one or more, two or more, three or more, four or more, five or more, six or more, seven or more, eight or more biomarkers in said samples, comparing the levels of said biomarkers to reference or normal levels, e.g., those observed in healthy subjects, comparing the levels of said biomarkers from the serial samples obtained from said subject, and detecting progression of HD in said subject when the levels of said biomarkers in the latest sample from said subject differ from levels in preceding samples obtained from the same subject, and treating said subject with a HD therapy for progression of HD. In one embodiment, said sample is plasma, said subject is a human, said serial samples are samples collected from the same subject at two or more time points separated by at least one or more, two or more, three or more, four or more, five or more six or more months. In one embodiment, said plasma biomarkers comprise N-methyl proline, beta-tocopherol, cortisone, trans-4-hyroxyproline, 2-hydroxyisobutyrate, butyrylglicine, phosphate, pyruvate, or any number of combinations thereof. Specifically, said differences in the levels of said plasma biomarkers are: N-methyl proline is about 5.8 raw peak intensity (log 10) for subjects with HD and is about 6.20 raw peak intensity (log 10) for healthy subjects, beta-tocopherol is ≧4.30 raw peak intensity (log 10) for subjects with HD and is ≦4.7 raw peak intensity (log 10) for healthy subjects, plasma cortisone is from about 5.35 to 5.45 raw peak intensity (log 10) for subjects with HD and is about 5.55 raw peak intensity (log 10) for healthy subjects, trans-4-hyroxyproline is about 6.8 raw peak intensity (log 10) for subjects with HD and about 6.55 to 6.95 raw peak intensity (log 10) for healthy subjects, 2-hydroxyisobutyrate is about 5.7 raw peak intensity (log 10) for subjects with HD and is about ≧5.65 raw peak intensity (log 10) for healthy subjects, butyrylglicine is about ≦5.2 raw peak intensity (log 10) for subjects with HD and is about 4.8 65 raw peak intensity (log 10) for healthy subjects, phosphate is about phosphate is about 7.93 to 8.6 raw peak intensity (log 10) for subjects with HD and about 8 to 8.18 raw peak intensity (log 10) for healthy subject, and pyruvate is about 5.7 to 5.9 raw peak intensity (log 10) for subjects with HD and about 5.7 to 6.4 raw peak intensity (log 10) for healthy subjects.

In other embodiments, said biomarkers of progression of HD are any one or any combination of biomarkers selected from those reported herein. The different levels of said plasma biomarkers are those reported herein.

In the present invention, a group of HD monkeys, which progressively develop motor and behavioral deficits, dystonia and chorea, were used in an exploratory longitudinal metabolomics study using plasma (and CSF, cross-sectionally) samples collected throughout the first two years of life (during which HD-related symptoms developed progressively). These symptoms distinctly paralleled those in humans with HD. This was the first longitudinal metabolomics profiling study using an animal model of HD. The study identified 27 individual plasma metabolites significantly (p<0.05) associated with HD progression over time.

Recent studies suggest that cognitive decline, psychiatric disturbances, and neuroanatomic as well as molecular profile changes may precede the appearance of motor deficits, making it of particular importance to identify early markers of disease pathogenesis (Kocerha, J, et al. Longitudinal transcriptomic dysregulation in the peripheral blood of transgenic Huntington's disease monkeys. BMC Neurosci. 2013; 14, 88; Aziz N A, et al. Autonomic symptoms in patients and pre-manifest mutation carriers of Huntington's disease. Eur J Neurol. 2010; 17, 1068-1074). HD monkeys provided a unique model system for investigating HD pathogenesis and progression through longitudinal approaches using clinical assessment tools comparable to those used in TRACK-HD and PREDICT-HD longitudinal studies of HD patients spanning the prodromal and manifest stages of the disease as it progresses.

### Measurements

Measurements of biomarkers may be made by an variety of methods known in the art such as by spectroscopic and chromatography methods, e.g., correlating to a specific molecular weight through mass spectroscopy and/or correlating to a specific retention or resolution time to pass through a chromatography medium, e.g., liquid chromatography, high-pressure liquid chromatography.

Flow cytometry is a laser based technique that may be employed in counting, sorting, and detecting biomarkers. A flow cytometer has the ability to discriminate different biomarkers on the basis of color. Differential dyeing of biomarkers emitting in different wavelengths, allows them to be distinguished.

In certain embodiments, the disclosure relates to a blood based method of identifying and measuring biomarkers which utilizes an analytical platform. Although the measurements are exemplified utilizing flow cytometry, in certain embodiments, the disclosure contemplates alternative methods such as using a solid surface array comprising probes, e.g., antibodies to the biomarkers. Provided herein are devices for detection of cells biomarkers with surfaces comprising, attached thereto, at least one reagent specific for one or more biomarkers.

In certain embodiments, the biomarkers can be immobilized to the surface by ligand binding and a detection reagent will bind specifically to the same biomarker. The detection reagent may be conjugated to an enzyme to generate a signal that can be quantified. For example, Rica & Stevens report an enzyme label that controls the growth of gold nanoparticles and generates colored solutions with distinct tonality when the analyte is present. See Nature Nanotechnology, 2012, 7:821-824.

In certain embodiments, a biomarker may be captured with a ligand or antibody specific for the biomarker which is covalently attached to a ligand or antibody capable of binding to a modified surface. In one example, a detection antibody conjugated to biotin or streptavidin—to create a biotin-streptavidin linkage to an enzyme that contains streptavidin or biotin respectively. A signal is generated by the conversion of the enzyme substrate into a colored molecule and the intensity of the color of the solution is quantified by measuring the absorbance with a light sensor. Contemplated assays may utilize chromogenic reporters and substrates that produce some kind of observable color change to indicate the presence of the biomarker. Fluorogenic, electrochemiluminescent, and real-time PCR reporters are also contemplated to create quantifiable signals.

Although some assay formats will allow testing of peripheral biological fluid samples without prior processing of the sample, it is typical that peripheral biological fluid samples will be processed prior to testing. Processing generally takes the form of elimination of cells, such as platelets in blood samples, and may also include the elimination of certain proteins, such as certain clotting cascade proteins from blood. In some examples, the peripheral biological fluid sample is collected in a container comprising EDTA.

The process of comparing a measured value and normal or a reference value can be carried out in any convenient manner appropriate to the type of measured value and reference value for the protein at issue. As discussed above, measuring can be performed using quantitative or qualitative measurement techniques, and the mode of comparing a measured value and a reference value can vary depending on the measurement technology employed. For example, when a qualitative calorimetric assay is used to measure biomarker levels, the levels may be compared by visually comparing the intensity of the colored reaction product, or by comparing data from densitometric or spectrometric measurements of the colored reaction product (e.g., comparing numerical data or graphical data, such as bar charts, derived from the measuring device).

The process of comparing may be manual (such as visual inspection by the practitioner of the method) or it may be automated. For example, an assay device (such as a luminometer for measuring chemiluminescent signals) may include circuitry and software enabling it to compare a measured value with a reference value. Alternately, a separate device (e.g., a digital computer) may be used to compare the measured value(s) and the reference value(s). Automated devices for comparison may include stored reference values for the protein(s) being measured, or they may compare the measured value(s) with reference values that are derived from contemporaneously measured reference samples.

In some embodiments, the methods of the disclosure utilize simple or binary comparison between the measured level(s) and the reference level(s) (e.g., the comparison between a measured level and a reference level determines whether the measured level is higher or lower than the reference level).

As described herein, biological fluid samples may be measured quantitatively (absolute values) or qualitatively (relative values). In certain aspects of the disclosure, the comparison is performed to determine the magnitude of the difference between the measured and reference values (e.g., comparing the fold or percentage difference between the measured value and the reference value).

### Juvenile or Adult Huntington's Disease

According to the Huntington's Disease Society of America Juvenile HD handbook (http://www.hdsa.org/living-with-huntingtons/publications/index.html#juvenile), the diagnosis of HD in an adult is usually made in a person who has memory or cognitive changes (dementia), and chorea (dance-like movements), often with behavioral or psychiatric problems such as depression, irritability, or mood swings, and usually with a family history of HD in a parent. The presenting symptoms may be a little different in a child, particularly a child under 10 years of age.

Most children affected with HD, and receiving diagnosis of HD, have at the time of onset, several of the following features: positive family history of HD in a parent, usually in the father, stiffness of the legs, clumsiness of arms and legs, decline in cognitive function, changes in behavior, seizures, changes in oral motor function, chorea in an adolescent, and behavioral disturbances. In the first decade of life, chorea is uncommon, but may be one of the first symptoms in a teenager. In an adolescent, the first symptoms of HD may be severe behavioral disturbances.

In the present invention, the non-human primate models of HD were monitored for motor and behavioral abilities from birth to full developmental maturity. Specifically, the HD and control primates were monitored for motor and behavioral abilities from birth to 36 months of age. The Juvenile Neurobehavioral Assessment Score was used to characterize the neuromotor abilities of juvenile HD and control non-human primates of 6 to 26 weeks of age, or from 1.5 to 6.5 months of age. Notable differences between the two groups were observed from two weeks onwards. These findings indicated that the non-human primate model of HD was useful in detecting reduced motor activity, poor orientation and neuromotor responses in juvenile HD monkeys, and the model recapitulated the symptoms used to diagnose juvenile HD in humans. Moreover, these findings indicated that the model was useful for obtaining and analyzing metabolites characteristic of onset and monitoring progression of juvenile HD.

### Non-Human Primates as Models for HD

The present invention relates to the methods of diagnosing onset and monitoring progression of neurological disorders, such as juvenile or adult HD, by measuring and monitoring levels of plasma and CSF biomarkers. The invention also relates to methods of initiation of therapies for HD in a subject with biomarker levels indicative of onset of HD. The invention further relates to methods of treating a subject with biomarker levels indicative of progression of HD with a HD therapy for progression of HD.

The metabolite levels of plasma and CSF biomarkers were measured in non-human primate models of HD. Because of the close physiological, neurological and genetic similarities between humans and higher primates (Lane M A. Nonhuman primate models in biogerontology. Exp. Gerontol. 2000; 35:533-541; King and Wilson. Evolution at two levels in humans and chimpanzees. Science. 1975; 188:107-116; McConkey and Varki. A primate genome project deserves high priority. Science. 2000; 289:1295-1296), monkeys can serve as very useful models for understanding human physiology and diseases (Chan A W, et al. Transgenic monkeys produced by retroviral gene transfer into mature oocytes. Science. 2001; 291:309-312).

Moreover, the symptoms of HD onset and progression in non-human primates very well recapitulate those of the human disease. For example, hallmark features of HD, including nuclear inclusions and neuropil aggregates, as well as clinical features of dystonia and chorea, were observed in the brains of the HD transgenic monkeys (Yang S H, et al. Towards a transgenic model of Huntington's disease in a non-human primate. Nature. 2008; 453(7197):921-924; Kocerha J, et al. Longitudinal transcriptomic dysregulation in the peripheral blood of transgenic Huntington's disease monkeys. BMC Neuroscience. 2013 Aug. 17; 14(1): 88).

Moreover, there was a longitudinal escalation in NDUFA5 transcript dysregulation with time and disease in non-human primates (Kocerha J, et al. Longitudinal transcriptomic dysregulation in the peripheral blood of transgenic Huntington's disease monkeys. BMC Neuroscience. 2013 Aug. 17; 14(1):88). Similarly, a recent study indicated that the mitochondrial respiratory chain, which NDUFA5 is part of, was dysfunctional in platelets from pre-symptomatic and symptomatic HD patients (Silva A C, et al. Mitochondrial respiratory chain complex activity and bioenergetic alterations in human platelets derived from presymptomatic and symptomatic Huntington's disease carriers. Mitochondrion. 2013; 51567-7249(13):00080-00089; Kocerha J, et al. Longitudinal transcriptomic dysregulation in the peripheral blood of transgenic Huntington's disease monkeys. BMC Neuroscience. 2013 Aug. 17; 14(1):88). Therefore, the changes in metabolite levels, detected in the non-human primate models of HD and provided in the current invention, are thought to be representative of the changes of these metabolites in the human subjects with HD.

Metabolomic Profiling of Plasma and/or CSF for Diagnosing Onset or Progression of HD

Metabolomics profiling studies are being used more and more in pursuit of novel disease biomarkers for HD (Casseb R F, et al. Thalamic metabolic abnormalities in patients with Huntington's disease measured by magnetic resonance spectroscopy. Braz J Med Biol Res. 2013; 46(8):722-727. Eckhart A D, et al. Metabolomics as a key integrator for “omic” advancement of personalized medicine and future therapies. Clin Transl Sci. 2012 June; 5(3):285-8. Lee D Y, et al. Mass spectrometry-based metabolomics, analysis of metabolite-protein interactions, and imaging. Biotechniques. 2010 August; 49(2):557-65. Quinones and Kaddurah-Daouk. Metabolomics tools for identifying biomarkers for neuropsychiatric diseases. Neurobiol Dis. 2009 August; 35(2):165-76).

Several reported metabolomics profiling studies have used different HD animal models; however, these studies were based largely on cross-sectional, not longitudinal, analysis of individuals. Cross-sectional studies are limited in that it is often difficult to control for environmental variables and truly isolate individual risk factors that inevitably play a role in HD onset and progression. The inventors used a large animal model that recapitulates the progressive clinical features of HD. This is a unique platform for identifying potential biomarkers for HD onset and therapeutic targets for developing treatments for HD. The current invention provides metabolite levels from plasma of HD and control non-human primate models obtained in a longitudinal study at different time points throughout the progression of the disease. The levels of biomarkers are provided for the time period of 5 months and 11 months of age, well in advance of the onset of the clinical signs of the disease at 17 months of age (Table 1). Also, the levels of biomarkers during the progression of the disease, at 17 months and 23 months of age, are also provided (Table 1). The present invention further provides metabolite levels from CSF of HD and control non-human primate models obtained in a cross-sectional study at one time point during the course of the disease, at onset of clinical symptoms of the disease (Table 2).

### Therapies at Onset and Progression of HD

In some embodiments, therapeutics to treat movement disorders include tetrabenazine (Xenazine).

In some embodiments, therapeutics to treat movement disorders include antipsychotic drugs, such as haloperidol (Haldol) and clozapine (Clozaril).

In some embodiments, therapeutics to treat movement disorders include other medications that may help suppress chorea, dystonia and muscle rigidity include antiseizure drugs such as clonazepam (Klonopin) and antianxiety drugs such as diazepam (Valium).

In some embodiments, therapeutics to treat psychiatric disorders will vary depending on the disorders and symptoms. In some embodiments, therapeutics to treat psychiatric disorders include antidepressants, such as escitalopram (Lexapro), fluoxetine (Prozac, Sarafem) and sertraline (Zoloft).

In some embodiments, treatment includes antipsychotic drugs that may suppress violent outbursts, agitation and other symptoms of mood disorders or psychosis. In some embodiments, treatment includes mood-stabilizing drugs that can help prevent the highs and lows associated with bipolar disorder include lithium (Lithobid) and anticonvulsants, such as valproic acid (Depakene), divalproex (Depakote) and lamotrigine (Lamictal).

In some embodiments, psychotherapy treatments include a psychotherapist—a psychiatrist, psychologist or clinical social worker—who can provide talk therapy to help a person with HD manage behavioral problems, develop coping strategies, manage expectations during progression of the disease and facilitate effective communication among family members.

In some embodiments, speech therapy includes exercises with a speech therapist to help improve the ability to speak clearly or teach to use communication devices—such as a board covered with pictures of everyday items and activities. Speech therapists can also address difficulties with muscles used in eating and swallowing.

In some embodiments, physical therapy includes appropriate and safe exercises that enhance strength, flexibility, balance and coordination for persons suffering from symptoms of HD. These exercises can help maintain mobility as long as possible and may reduce the risk of falls. Instruction on appropriate posture and the use of supports to improve posture may help lessen the severity of some movement problems. Also, exercise regimens can be adapted to suit the new level of mobility.

In some embodiments, occupational therapy with an occupational therapist can assist the person with HD, family members and caregivers on the use of assistive devices that improve functional abilities. These strategies may include handrails at home, assistive devices for activities such as bathing and dressing, and eating and drinking utensils adapted for people with limited fine motor skills.

### EXAMPLES

### Neurobehavioral Assessment of HD Monkeys

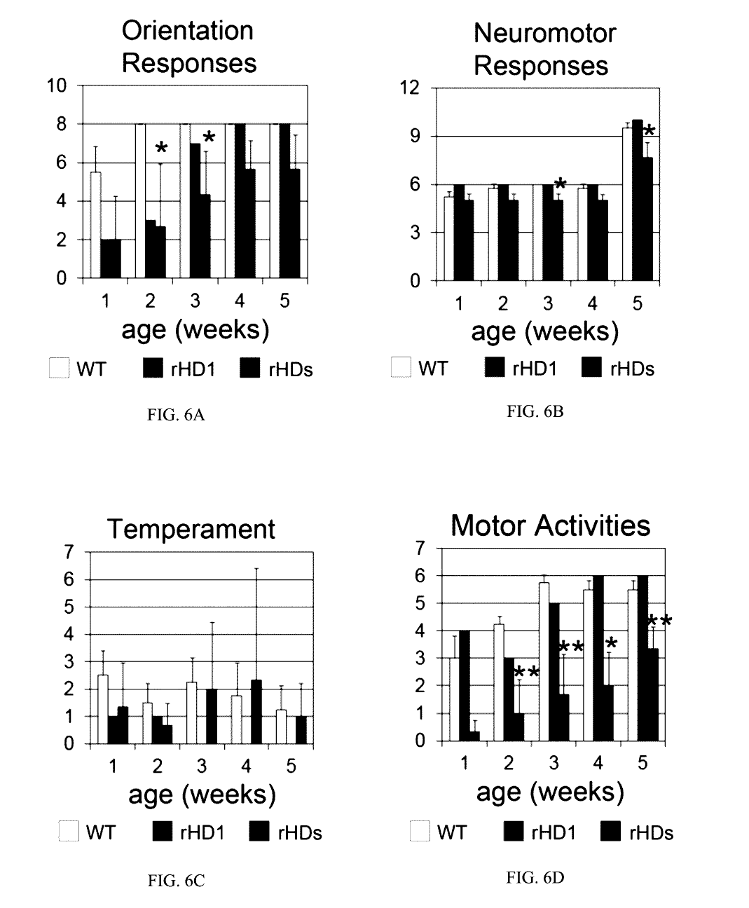

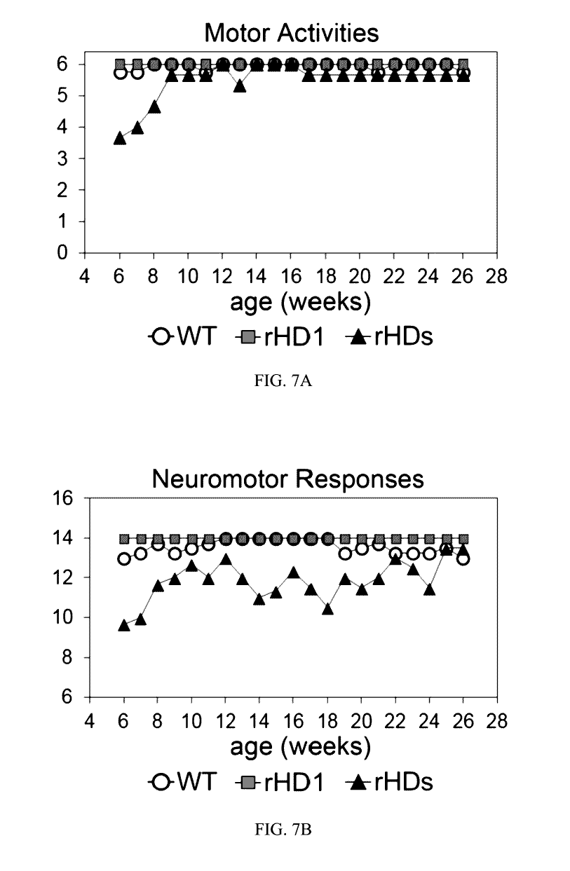

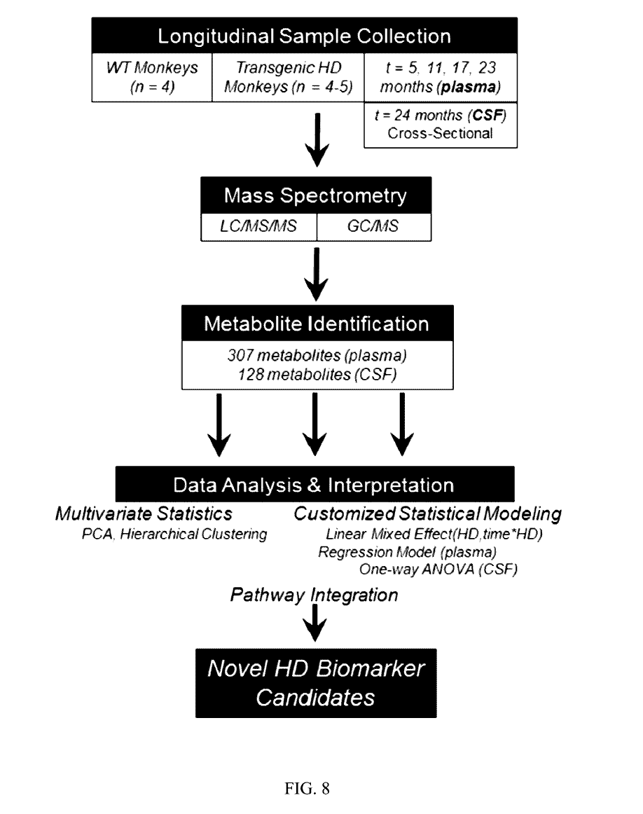

A group of HD monkeys (n=4; rHD1, rHD6, rHD7 and rHD8) and a group of WT monkeys (n=4; two males and two females) were enrolled in a longitudinal study that encompassed cognitive behavioral assessment and metabolomics profiling in plasma and CSF. rHD1, as described previously (Yang S H, et al. Towards a transgenic model of Huntington's disease in a non-human primate. Nature. 2008; 453, 921-924), carried exon 1 of the human huntingtin (HTT) gene driven by a human polyubiquitin promoter, with 29 polyglutamine (polyQ) repeats at the N-terminus. Rhesus macaques (WT) normally carry 10-11 polyQ repeats. rHD6, 7 and 8 (rHDs) carry exon 1-10 of the human HTT gene driven by human HTT promoter with 67Q, 70Q and 72Q at the N-terminus, respectively (Kocerha, J, et al. Longitudinal transcriptomic dysregulation in the peripheral blood of transgenic Huntington's disease monkeys. BMC Neurosci. 2013; 14, 88). All monkeys were characterized and evaluated via longitudinal assessment of neurobehavioral and motor functions throughout development. HD monkeys gradually developed motor deficits and other features similar to those seen in HD patients. Different behavioral tests in each of the HD and WT monkeys were performed at various ages, spanning from birth to three years of age. During the first five weeks postpartum, orientation response, neuromotor response, temperament, and motor response using the Infant Neurobehavioral Assessment Scale (INAS) were assessed (FIG. 6). Strength in orientation responses increased rapidly in controls, with levels plateauing at week 2, whereas the HD monkeys displayed weaker visual and auditory reflexes that slowly improved with time. Although orientation responses in rHD1 reached control levels by week 4, the orientation responses of rHD6, rHD7 and rHD8 remained at lower levels that in the WT monkeys for weeks 2 and 3 (HD<controls: U=2.0, p=0.04, respectively; FIG. 6 panel a). Neuromotor responses and motor activity of rHD1 were comparable to levels seen in controls; the other three HD monkeys exhibited slightly reduced neuromotor responses at weeks 3 and 5 (U=2, p<0.04 and U=1, p<0.04, respectively), as well as poorer motor control at all weeks (all p-values <0.03 for weeks 2-5; FIG. 6 panel b and 6 panel d). Temperament measures were comparable amongst the WT and HD monkeys (FIG. 6 panel c). A similar assessment was performed from weeks 6 to 26 (Juvenile Neurobehavioral Assessment Scale; JNAS). Although rHD1 continued to show normal motor development during the juvenile period, the motor development of the remaining three HD monkeys lagged behind controls. Motor activity in HD monkeys improved steadily through weeks 6-12, ultimately reaching normal locomotor ability [group×week: F(20, 120)=12.46, p<0.002] (FIG. 7 panel a). Interestingly, the neuromotor responses of rHD6, rHD7 and rHD8 improved slowly with age, but never reached the strength seen in controls [group effect: F(1, 6)=12.46, p<0.01] (FIG. 7 panel b).

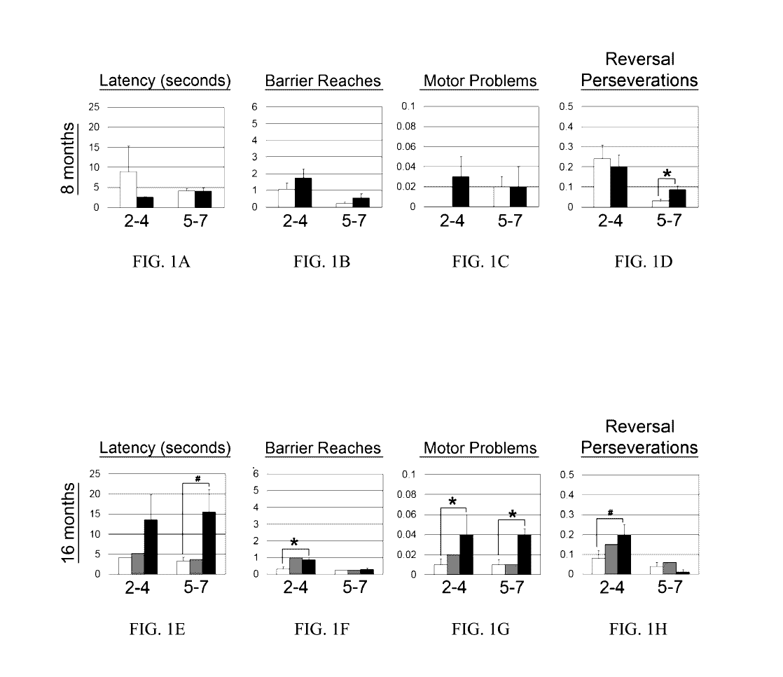

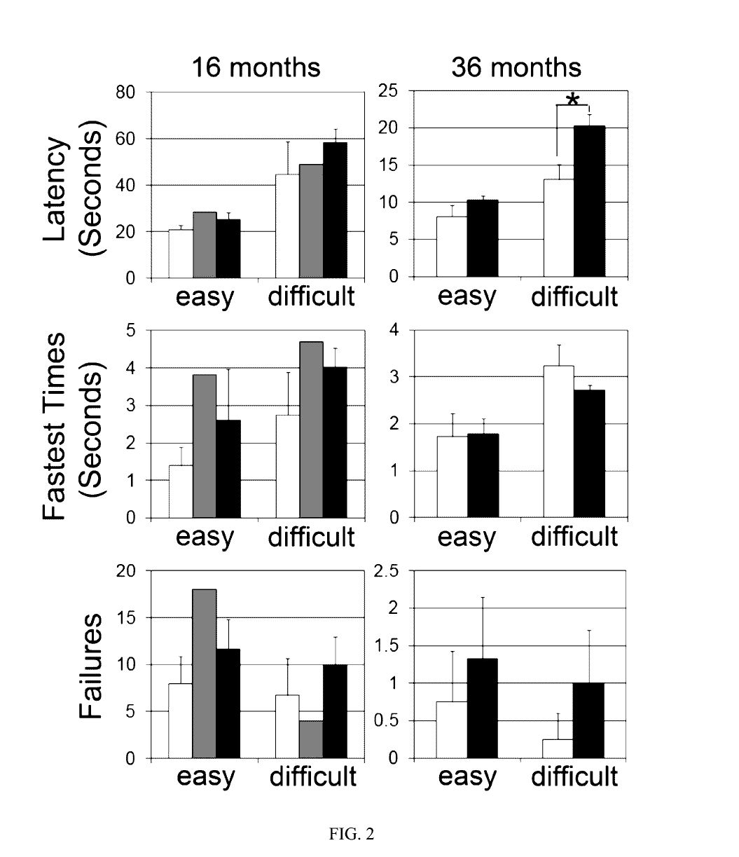

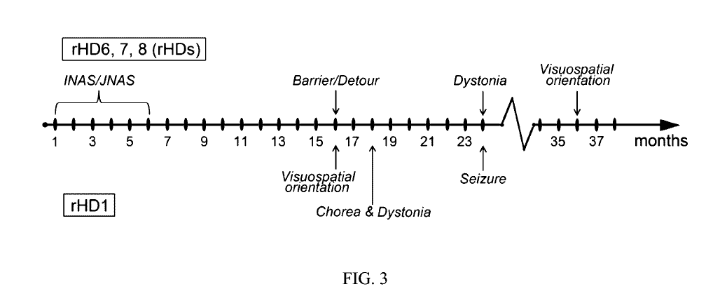

At both 8 and 16 months of age, animals performed the detour/barrier task. Performance results at 8 months of age were similar between HD monkeys and WT monkeys (FIG. 1 panel a-d; top panel), although HD monkeys displayed a slightly higher number of perseverations in the difficult trials (FIG. 1 panel d) [t=3.315 with 5 d.f., p=0.021] (rHD1 was not assessed at 8 months of age). Notably, testing conducted on HD monkeys at 16 months of age revealed a gradual impairment in motor function (FIG. 1 panel e-h; bottom panel). It is interesting that rHD1 showed no impairment in the task at 16 months of age, whereas problems gradually emerged in the other three HD monkeys. rHD6, rHD7 and rHD8 took longer to retrieve the rewards than controls in both the easy (Trials 2-4) and difficult trials (Trials 5-7), although this group difference showed only a trend towards significance for the difficult trials [t(5)=2.34, p=0.06] (FIG. 1 panel e); they also performed more barrier reaches [t(5)=2.60, p=0.048] (FIG. 1 panel f) and tended to have more perseverative errors [t(5)=2.21, p=0.07] (FIG. 1 panel h) in the easy trials. They displayed significantly greater motor problems when attempting to retrieve the food rewards in all trials [U=0.5, p=0.04 and U=0, p=0.026, for easy and difficult trials, respectively; FIG. 1 panel g]. Furthermore, visuomotor performance was assessed at both 16 and 36 months of age using the Visuospatial Orientation (VS-OR; Life Saver) task. At 16 months of age, there were no significant differences between the HD and WT monkeys in any of the parameters tested (latency, fastest times and failures; FIG. 2, left panel). At 36 months of age, however, HD monkeys required more time to perform the task when more difficult patterns were used [t(5)=3.73, p=0.014, for latency]. Note that testing of rHD1 in the Life Saver task was discontinued due to the development of self-injury behavior during testing. Together, the detour/barrier and Life Saver tasks revealed that HD monkeys developed motor dysfunction and lack of behavioral inhibitory control, both symptoms associated with HD onset/progression in humans. Additionally, progressive motor and behavioral impairment in rHD1 differed from the other three HD monkeys (rHD6, rHD7 and rHD8), suggesting the influence of transgene constitution between the two groups. The timeline of progressive development of behavioral impairment between the two groups of HD monkeys is illustrated in FIG. 3.

### Metabolic Defects in HD Monkeys

Having seen a progressive manifestation of HD-like symptoms in the HD monkeys, the study focused on whether any abnormalities in metabolism were detectable during the presumed period of onset. Cross-sectional metabolomics profiling study in the HD and WT monkeys were performed using CSF samples collected at 24 months of age. A total of 128 metabolites were positively detected by mass spectrometry, and the resulting data were analyzed by principal components analysis (PCA) and one-way analysis of variance (ANOVA).

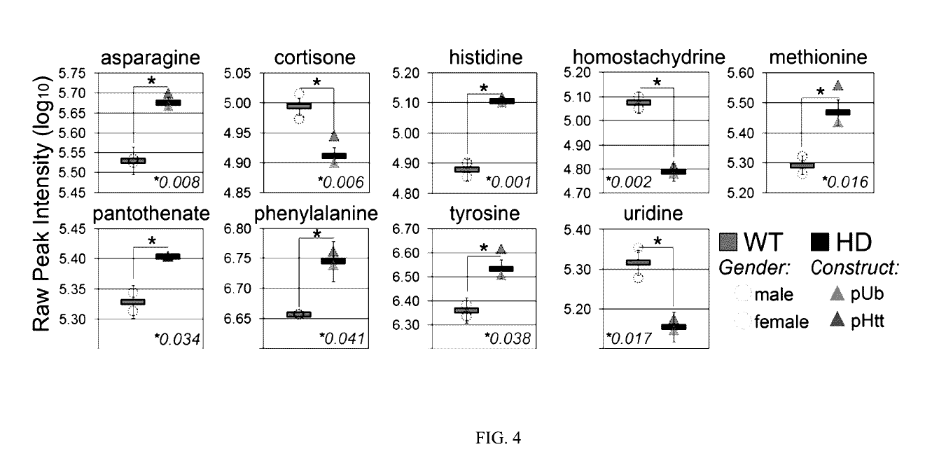

The dimensionality of the CSF metabolomics dataset was assessed by performing PCA, a multivariate statistical method that summarizes the structure of large datasets as orthogonal linear combinations of the variables that best explain the overall variation. Principal components (Prins) were generated from the analysis; the greatest percentage of total variance accounted for by any projection of data lies on Prin1, the second greatest on Prin2, and so on. PCA was performed on the CSF metabolite data and resulting Prins were plotted in a 3-dimensional (3-D) scatterplot (FIG. 4 panel a). Normalized ellipsoid contours were highlighted in the graph, which grouped results by disease status (HD—red or WT—green). A substantial amount of variance was apparent in the data within each treatment group, but the experimental factors responsible for the greatest variance in the dataset were not immediately clear. Prin1, Prin2 and Prin3 accounted for 32.4, 20.3 and 13.6% of the variance, respectively. Notably, HD and WT monkey data clustered separately in the metabolomics dataset, implying the early-stage HD phenotype may be discernable in the CSF metabolite profiles of the HD monkeys.

To refine the metabolomics dataset and determine whether nine individual metabolites exhibited significantly different levels based on disease status (HD vs. WT, p<0.05), one-way ANOVA was performed. Using only the enriched data, a more focused PCA was performed. Results were plotted in 3-D in FIG. 4 panel b, with each treatment group highlighted as in FIG. 4 panel a. Disease status very clearly corroborated with Prin1, which accounted for 72.4% of the variance in the enriched dataset. Data for the nine CSF metabolites displayed a strong correlation with HD.

All nine significant CSF metabolites are plotted in FIG. 4 panel c. Most of the metabolites were identified as amino acids and amino acid derivatives, including histidine, methionine, homostachydrine (proline-betaine) and asparagine, as well as the dopamine precursors tyrosine and phenylalanine. Levels of these amino acids were higher in the CSF of HD monkeys compared to controls, except for homostachydrine levels, which were lower. Pantothenate (vitamin B5), a coenzyme A constituent, was elevated in the CSF of the HD monkeys, whereas uridine (pyrimidine metabolism) and cortisone (cortisol metabolite) levels were significantly decreased in the CSF of the HD monkeys. Overall, the cross-sectional CSF metabolomics analysis of the present invention indicated that CSF metabolite levels, especially amino acid metabolites, are different between the HD and WT monkeys. The CSF metabolic signatures of HD and WT monkeys were distinguishable at 24 months of age.

### Longitudinal Plasma Metabolomics Profiling

An exploratory metabolomics profiling study using plasma samples longitudinally collected from the HD and WT monkeys spanning the first two years of age (5, 11, 17 and 23 months) was performed; based on the behavioral assessment described above, this time frame was expected to encompass the prodromal and early manifest stages of HD in the HD monkeys. Multivariate statistical analyses were used to examine the sources of variance within the data; specifically, we examined whether any known variable in the design of this study (disease status, age, gender, construct) might be a source of variance in the results.

PCA was performed, and the first three Prins are graphically displayed in 3-D scatterplots for adequate visualization of results. The first three Prins accounted for 19.5, 13.5 and 8.74%, respectively, of the total variation seen in the plasma metabolite data. Normalized ellipsoid contours were superimposed in the 3-D PCA scatterplot to examine whether disease status (HD or WT) could account for any of the observed variance. Initial PCA results from the longitudinal plasma metabolomics dataset failed to show a distinct difference between HD and WT data.

PCA results were then examined for age-specific variation, and data were superimposed with ellipsoids that highlighted age. Analysis revealed that age (longitudinally) was a distinguishable source of a large amount of variation in the plasma metabolite data. Differentially colored ellipsoids in the figure highlight each time point/age at which samples were profiled. Data collected at 5 months showed substantial overlap with data collected at 11 months, and data collected at 17 and 23 months exhibited the same overlap in results. Data collected at 5 and 23 months were most visibly different.

The initial PCA results suggested that the age of subjects associated with more of the variation in the metabolomics data than did disease status, implying that a statistical approach encompassing the longitudinal nature of our study would be necessary to account for the age-specific variation in the overall dataset.

To more stringently examine the plasma metabolite results for any putatively novel HD biomarkers, data were fit to a custom mixed-effect linear regression model that accounted for our longitudinal data structure. This model accounted for time, but tested data specifically for significant differences between HD and WT monkeys based on the combined joint effect of disease status (HD) and disease status×time (HD,HD×time). The mixed-effect model identified 27 plasma metabolites exhibiting nominally significant (p<0.05) and divergent trends between the HD and WT monkey groups over time.

Profiling data for the plasma metabolites (n=27, p<0.05) identified by the custom linear model (enriched plasma metabolite data) were then subjected to PCA (focused PCA) to validate whether disease status and time contributed to an enriched amount of variation amongst the focused dataset. Both disease status and age (months) were distinct sources of the highest variation within the enriched dataset. The first principal component (Prin1) accounted for 24.9% of the variation in the data, while the second (Prin2) accounted for an additional 17.2%. Prin1 and Prin2 combined accounted for ˜40% of the variation observed within the data from the significant plasma metabolites when all time points were considered together.

PCA was subsequently run using the significant metabolite data (focused PCA by age), but data were sorted into four individual subsets by age prior to performing PCA on each data subset independently. Graphs represent focused PCA results from analysis of the 27 significant metabolites at 5, 11, 17 and 23 months of age. Normal contour ellipsoids encompass disease status amongst the data; red highlights the HD group, and green highlights WT monkey data. Prin1 was clearly defined by disease status, and over time the percentage of variation coverage seen in the first component increased from 29.6% at 5 months of age to nearly 50% at 17 and 23 months of age, suggesting disease progression may occur as the animal ages.

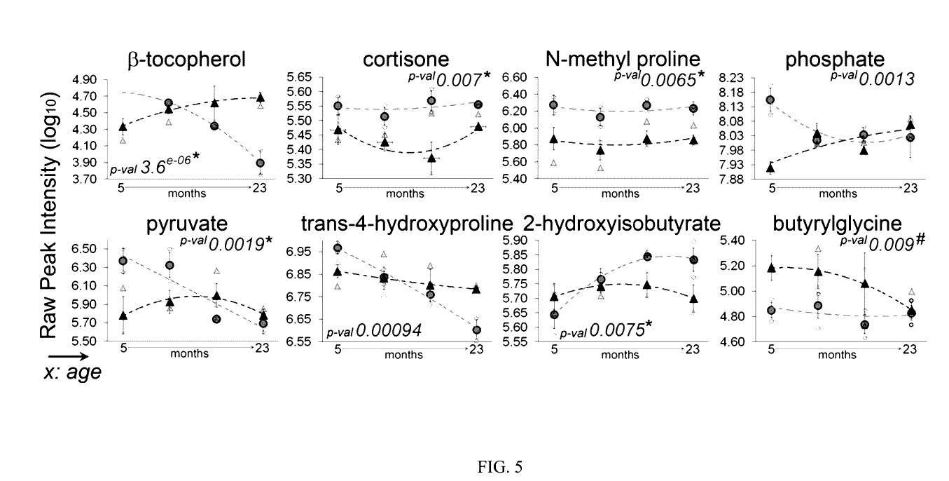

Of the 27 metabolites that exhibited significantly (p<0.05) divergent trends between HD and WT monkey groups, eight metabolites possessed the highest potential as early HD biomarkers, with joint p-values <0.01 (FIG. 5). Focused PCA was performed (by age) using only the enriched data for the eight biomarker candidates, and results were plotted in 3-D scatterplots by age (FIG. 5 panel a). Ellipses encompassing disease status (HD—red or WT—green) highlight the visual clustering of the data between the HD and WT monkeys. Metabolite levels were plotted individually for each of the eight HD biomarker candidates, displaying divergence between the HD and WT cohorts with age (FIG. 5 panel b). Among them, beta-tocopherol, pyruvate, phosphate and trans-4-hydroxyproline were most significantly divergent, with joint p-values <0.002.

An overall examination of the metabolites found to be significantly associated with early-stage HD in HD monkeys revealed a prominence in amino acid metabolites in plasma (and CSF), as well as an enriched dysregulated number of plasma metabolites involved in lipid metabolism.

### Methods Summary

All animal procedures (e.g. housing, bleeding) were performed in a BSL-2 facility at the Yerkes National Primate Research Center (YNPRC) and were approved by the Emory University Institutional Animal Care and Use Committee (IACUC). Transgenic HD monkeys were created by lentiviral transfection of rhesus mature oocytes, followed by in vitro fertilization, culture and embryo transfer into surrogate female. Behavior and neurological evaluation were performed and monitored by the research staff and veterinary staff during experimental procedures and on a routine basis. Blood and CSF were collected and processed from all HD and WT monkeys following an approved protocol. Schematic illustration of methods for metabolomics profiling of plasma and CSF are presented in FIG. 9.

What is claimed is:

1. A method of diagnosing onset or progression of Huntington's disease (HD) comprising:
obtaining a sample from a subject,
measuring levels of at least one biomarker in said sample, wherein the at least one biomarker comprises one of N-methyl proline or beta-tocopherol;
comparing the levels of said biomarkers to a reference value, and
diagnosing said subject with an onset of HD when the levels of said biomarkers from said subject differ from those of the reference value and treating said subject with a HD therapy.

2. The method of claim 1, wherein said sample is plasma and/or cerebrospinal fluid (CSF).

3. The method of claim 1, wherein said subject is a human.

4. The method of claim 1, wherein measuring levels of biomarkers in said sample comprise measuring the levels of at least two of the biomarkers.

5. The method of claim 4, wherein said biomarkers include cortisone.

6. The method of claim 5, wherein said biomarkers are beta-tocopherol, pyruvate, phosphate and trans-4-hydroxyproline.

7. The method of claim 1, wherein said plasma biomarkers are N-methyl proline, cortisone, beta-tocopherol, trans-4-hyroxyproline, 2-hydroxyisobutyrate, butyrylglicine, phosphate, pyruvate, or any number of combinations thereof.

8. The method of claim 1, wherein said CSF biomarkers are cortisone, asparagine, histidine, homostachydrine, methionine, pantothenate, phenylalanine, tyrosine, and uridine, or any number of combinations thereof.

9. The method of claim 1, wherein measuring levels of said biomarkers comprise utilizing mass spectrometry, enzyme-linked immunosorbent assay (ELISA), high-performance liquid chromatography (HPLC), or any variation thereof, to obtain the levels of said biomarkers in said samples.

10. A method of monitoring progression of HD, comprising
obtaining at least two serial samples from a subject,
measuring the levels of at least one or more biomarkers in said samples,
comparing the levels of said biomarkers from said serial samples obtained from said subject, and
detecting progression of HD in said subject when the levels of said biomarkers from said serial samples from said subject differ, and when the levels of said biomarkers from latest serial sample from said subject differs from those observed in the preceding sample obtained from the same subject, and treating said subject with HD therapy when the progression is detected.

11. The method of claim 10, wherein said sample is plasma.

12. The method of claim 10, wherein said two serial samples are at least a first sample and a second sample collected from the same subject at two time points separated by at least four or more months.

13. The method of claim 10, wherein said plasma biomarkers are beta-tocopherol, pyruvate, phosphate and trans-4-hydroxyproline.

14. The method of claim 10, wherein said plasma biomarkers are N-methyl proline, beta-tocopherol, cortisone, trans-4-hyroxyproline, 2-hydroxyisobutyrate, butyrylglicine, phosphate, pyruvate, or any number of combinations thereof.
